(19) United States
(12) Patent Application Publication
(10) Pub. No.: US 2010/0035838 A1
(43) Pub. Date: Feb. 11, 2010

(54) Cross-linked polysaccharide gels

(76) Inventors: Geoffrey Kenneth Heber, Annandale (AU); Nicholas Patrick John Stamford, Wahroonga (AU)

Correspondence Address: O'BRIEN JONES, PLLC, 8200 Greensboro Drive, Suite 1020A, McLean, VA, 22102, US

(21) Appl. No.: 12/442,073

(22) PCT Filed: Sep. 18, 2007
(86) PCT No.: PCT/AU2007/001378

§ 371 Date: Sep. 10, 2009

(30) Foreign Application Priority Data
Sep. 19, 2006 (AU) 2006905174

(51) Int. Cl. A61K 31/715; C07H 1/00; A61P 43/00

(52) U.S. Cl. 514/54; 536/123.1

(57) ABSTRACT

The present invention relates to a process for preparing a cross-linked polysaccharide gel comprising contacting a polysaccharide with a cross-linking agent and a masking agent to form a cross-linked polysaccharide gel having resistance to degradation under physiological conditions.

### TECHNICAL FIELD

The present invention relates to cross-linked polysaccharide gels, processes for preparing the gels, and uses of the gels in cosmetic, medical and pharmaceutical applications.

### BACKGROUND ART

The dermis lies between the epidermis and the subcutaneous fat and is responsible for the thickness of the skin and, as a result, plays a key role in skin's cosmetic appearance. Fibroblasts are the primary cell type in the dermis and produce collagen, elastin, other matrix proteins and enzymes, such as collagenase and hyaluronidase. Elastin fibrils, collagen fibrils and hyaluronic acid (HA) are known to associate using non-covalent bonds, lending structure to the skin. These interactions are disturbed in aged skin, likely because of the decreased amount of (HA) in aged skin.

HA, also known as hyaluronan, is the most abundant non-sulfated glycosaminoglycan component of the human dermis. Although the primary function of HA in the intercellular matrix is to provide stabilization to the intercellular structures and to form the elastoviscous fluid matrix in which collagen and elastin fibers are embedded, HA is also important in cell growth, membrane receptor function and adhesion. The structure of HA is identical regardless of whether it is derived from bacteria, animals or humans.

The concept of using HA as a dermal filler was first developed due to the biocompatibility and lack of immunogenicity of HA. As such, HA is an attractive building block for new biocompatible and biodegradable polymers that have applications in drug delivery, tissue engineering, and viscosupplementation. However, the development of new biomaterials is precluded by the poor biomechanical properties of HA.

HA has a large molecular weight and is made of repeating dimers of glucuronic acid and N-acetyl glucosamine assembled into long chains. These chains form highly hydrated random coils, which entangle and interpenetrate each other producing highly elastoviscous solutions. However, unmodified, natural state HA has an extraordinarily high rate of turnover in vertebrate tissues and is rapidly broken down by hyaluronidase, β-D-glucuronidase and β-N-acetyl-D-hexoaminidase. In skin, the half life of unmodified HA is 12 hours, and in the bloodstream, 2 to 5 minutes.

A variety of chemical modifications of native HA have been devised to provide mechanically and chemically robust derivative materials. The resulting HA derivatives have physicochemical properties that may significantly differ from the native polymer, but most derivatives retain the biocompatibility and biodegradability, and in some cases the pharmacological properties, of native HA.

The prototypical modification is conversion of the viscous form to a cross-linked hydrogel by chemical cross-linking of polymers to infinite networks. This modification has been accomplished under mild, neutral conditions and under alkaline conditions. Indeed, these water-binding gels (hydrogels) are now widely used in the biomedical field and several cross-linked HA products are currently on the market as dermal fillers.

Injectable hydrogels have been prepared from HA which have a zero, low or high degree of cross-linking. The cross-linking of the polymer is usually effected in the presence of an agent such as aldehydes, bisepoxides, polyaziridyl compounds and divinylsulfone.

The most often utilised cross-linking agents are the polyepoxides (in particular 1,4-butanediol diglycidyl ether (or 1,4-bis(2,3-epoxypropoxy)butane or 1,4-bisglycidyloxybutane=BDDE), 1,2-bis(2,3-epoxypropoxy)ethylene and 1-(2,3-ep-oxypropyl)-2,3-epoxycyclohexane). In these cases, the cross-linking agent usually forms cross-links in polysaccharides via their hydroxyl groups and are usually performed by reacting a controlled amount of the cross-linking agent with the HA polymer dissolved in a basic medium.

Hyaluronidase itself is an endo-glycosidase (an enzyme that cleaves internal to HA polymers). More importantly, solution-binding studies on the testicular derived enzyme have shown that (GlcA-GlcNAc)₃ is the smallest oligomer that can be hydrolysed. In the case of the bee venom enzyme, hyaluronidase cleaves between the −1 and +1 sites and the −1 sugar is distorted toward the transition state for this reaction. The residue Glu113 of the enzyme acts as the catalytic acid and the catalytic nucleophile is presumably the N-acetyl function of the sugar. Human hyaluronidase has also been shown to have remarkable sequence similarity to that of the bee venom enzyme with regard to these active site regions.

For every repeating disaccharide in the HA chain there are 4 hydroxyl groups available to form an ether link with an epoxide of BDDE. It has been previously shown that hyaluronidase requires 6 sugars (3 disaccharides) for effective binding to the polysaccharide.

It might therefore be assumed that the chemical modification of the HA backbone at intervals may impart some degree of inability in the capacity of the hyaluronidase to recognise, appropriately bind, and/or catalyse the cleavage of HA oligomers. In this light it is quite reasonable to expect that it is not the formation of cross-links per se that masks the HA to recognition and subsequent cleavage by the hyaluronidase and engenders partial resistance to HA-based hydrogels, but rather the repeated modification of the HA itself.

The present inventors have produced cross-linked polysaccharide gels having a higher proportion of ether-links which results in new hydrogels having improved degradation characteristics.

### DISCLOSURE OF INVENTION

In a first aspect, the present invention provides a process for preparing a cross-linked polysaccharide gel comprising:

contacting a polysaccharide with a cross-linking agent and a masking agent under conditions to form a cross-linked polysaccharide gel having resistance to degradation under physiological conditions.

Preferably, the polysaccharide is contacted with the cross-linking agent and the masking agent under alkaline conditions to form a cross-linked polysaccharide substantially linked by ether bonds.

Preferably, the process further comprises:

drying the cross-linked polysaccharide without substantially removing the cross-linking agent or the masking agent to form a cross-linked polysaccharide matrix; and

neutralising the cross-linked polysaccharide matrix with an acidic medium to form the cross-linked polysaccharide gel.

Preferably the process further comprises:

washing the cross-linked polysaccharide gel with a water-miscible solvent.

Advantageously, it has been determined that when the cross-linked gel is formed by the process according to the present invention, the gel has improved resistance to degradation in situ when compared to conventional cross-linked polysaccharide gels.

A variety of different polysaccharide starting materials may be used in the present invention. Examples include, but are not limited to, the polysaccharide is selected from hyaluronic acid, chondroitin sulphate, heparin, starch, maltodextrins, cellodextrins, cellulose, chitosan, glucomannan, pectin, xanthan, algiinic acid, carboxymethyl cellulose, carboxymethyl dextran, carboxymethyl starch and carrageenans. Preferably, the polysaccharide is HA.

A variety of cross-linking agents may be used in the present invention. Examples include, but not limited to, aldehydes, epoxides, glycidyl ethers, polyaziridyl compounds and divinylsulfones. Preferably, the cross-linking agent is ethylene glycol diglycidyl ether, 1,4-butanediol diglycidyl ether (BDDE), 1,4-bis(2,3-epoxypropoxy)butane, 1,4-bisglycidyloxybutane, 1,2-bis(2,3-epoxypropoxy)ethylene, or 1-(2,3-epoxypropyl)-2,3-epoxycyclohexane. Preferably the cross-linking agent is a bis-functional epoxide. More preferably, the cross-linking agent is 1,4-butanediol diglycidyl ether (BDDE). It will be appreciated, however, that other cross-linking agents may also be suitable for the present invention.

A variety of masking agents may be used in embodiments of the present invention. Examples include, but are not limited to, ethylene oxide, propylene oxide, ethyl vinyl sulfone, methyl vinyl sulfone, or glycidol. The masking agent is preferably a mono-functional epoxide. More preferably, the masking agent is glycidol, or ethyl vinyl sulfone. Even more preferably, the masking agent is glycidol. It will be appreciated, however, that other masking agents may also be suitable for the present invention.

The polysaccharide starting material is typically combined with the cross-linking agent in an alkaline medium. In one embodiment, between about 1 and about 10 w/v percent, more particularly about 4 w/v percent, polysaccharide may be added to the alkaline medium. The alkaline medium may be formed with sodium hydroxide or other suitable basic materials such as potassium hydroxide or various organic and inorganic bases. The concentration of sodium hydroxide or other basic material may be between about 0.1 and about 1 w/v percent, more particularly about 1% of the total mixture. The cross-linking agent is typically added to the alkaline mixture to provide a cross-linking agent at a concentration between about 0.05 and about 1.0% (w/v), more particularly about 0.1% (w/v). The alkaline medium may have a pH between about 8 and 14, more particularly, about 9.

The resulting alkaline mixture may be incubated under conditions that promote cross-linking of the polysaccharide with the masking agent. For example, the mixture may be incubated in a water bath at about 45° C. for about 2 hours. Other temperatures such as 0-100° C. would also be suitable.

After incubation, the cross-linked polysaccharide is typically dried by conventional methods to form a polysaccharide matrix. For example, the cross-linked polysaccharide may be dried by stirring vigorously and removing water present under high vacuum for about 20 to 40 mins, up to 1 hour at between about 35° C. and 45° C. Other temperatures such as 0-100° C. would also be suitable. After drying, the polysaccharide matrix is typically neutralised with an acidic medium to form a cross-linked polysaccharide gel. For example, the matrix may be treated with a solution of about 1 to 3% acetic acid in water to neutralize the formed cross-linked polysaccharide gel. The polysaccharide gel may be washed with a water miscible solvent, for example an isopropyl alcohol/water co-solvent, for several hours. Polysaccharide such as HA cross-linked under these conditions will substantially include ether bonds which are generally more resistant to physiological degradation than ester bonds formed under acidic conditions.

As further set out in the Examples below, the polysaccharide gel formed by the method of the present invention is sufficiently cross-linked to resist degradation when administered to a patient or subject. Because of the improved degradation characteristics of the cross-linked polysaccharide gel, the gel may be used for a variety of applications. For example, the cross-linked polysaccharide gel may be used for augmenting tissue, treating arthritis, treating tissue adhesions, and for use in coating mammalian cells to reduce immunogenicity. Furthermore, the cross-linked polysaccharide gel may be used in cosmetic applications, corrective implants, hormone replacement therapy, hormone treatment, contraception, joint lubrication, and ocular surgery.

Advantageously, the cross-linked polysaccharide gel remains substantially resistant to degradation following extrusion through a narrow gauge needle. Extrusion through a needle may break gels into smaller particles if the gels are not resistant to shear stress. In particular, the cross-linked polysaccharide gels of the present invention are resistant to degradation following extrusion through a small gauge needle such as a 27, 30 or 32 gauge needle. Thus, these gels are particularly suitable for injection into tissue or skin without substantial loss of the structural integrity of the solution or gel.

In a preferred form, the present invention provides a process for preparing a cross-linked hyaluronic acid gel comprising:

(a) contacting hyaluronic acid under alkaline conditions with a cross-linking agent and a masking agent to form a cross-linked hyaluronic acid substantially linked by ether bonds;
(b) drying the cross-linked hyaluronic acid without substantially removing the cross-linking agent or the masking agent to form a cross-linked hyaluronic acid matrix; and
(c) neutralising the cross-linked hyaluronic acid matrix with an acidic medium to form a cross-linked hyaluronic acid gel having resistance to degradation under physiological conditions.

Preferably the process further comprises:

(d) washing the cross-linked hyaluronic acid gel with a water-miscible solvent.

Preferably, the ether bonds are formed about every three disaccharide units of the hyaluronic acid.

Preferably, the cross linking agent is a bis-functional epoxide. More preferably the cross-linking agent is 1,4-butanediol diglycidyl ether (BDDE). Preferably the masking agent is a mono-functional epoxide. More preferably, the masking agent is glycidol.

In a second aspect, the present invention provides a cross-linked polysaccharide gel substantially resistant to hyaluronidase degradation under physiological conditions prepared by the process according to the first aspect of the present invention.

In a third aspect, the present invention provides a cross-linked polysaccharide gel comprising hyaluronic acid cross-linked substantially by ether bonds with a cross-linking agent and a masking agent such that the gel is sufficiently cross-linked to have resistance to degradation under physiological conditions.

Preferably, the gel is substantially resistant to degradation by hyaluronidase under physiological conditions.

In a fourth aspect, the present invention provides a pharmaceutical composition comprising a cross-linked polysaccharide gel according to the second or third aspects of the present invention, a biologically active substance, and a pharmaceutically acceptable carrier.

The cross-linked polysaccharide gel according to the present invention may be combined with a biologically active substance for administration to a patient or subject. Suitable biologically active substances for use with the present invention include hormones, cytokines, vaccines, cells, tissue augmenting substances, or mixtures thereof. Examples of suitable tissue augmenting substances include collagen, starch, dextranomer, polylactide, poly-beta-hydroxybutyrate, and/or copolymers thereof.

The biologically active substance may be combined with suitable cross-linked polysaccharide gels of the present invention by physical mixing of the biologically active substance with the polysaccharide starting material. The biologically active substance may be combined in solid form, for example as a freeze-dried powder or solution.

In certain embodiments, the biologically active gels may be formed into pharmaceutical preparations for oral, rectal, parenteral, subcutaneous, local or intradermal use. Suitable pharmaceutical preparations may be in solid or semisolid form, for example pills, tablets, gelatinous capsules, capsules, suppositories or soft gelatin capsules. For parenteral and subcutaneous uses, pharmaceutical preparations intended for intramuscular or intradermal uses or infusions or intravenous injections may be used, and may therefore be presented as solutions of the active compounds or as freeze-dried powders of the active compounds to be mixed with one or more pharmaceutically acceptable excipients or diluents. Additionally, pharmaceutical preparations in the form of topical preparations may be suitable, for example nasal sprays, creams and ointments for topical use or sticking plasters specially prepared for intradermal administration.

In a fifth aspect, the present invention provides a method of augmenting skin comprising administering to a patient a cross-linked polysaccharide gel according to the second or third aspects of the present invention.

In a sixth aspect, the present invention provides a method of treating or preventing a disorder in a subject in need thereof comprising administering a therapeutically effective amount of a pharmaceutical composition according to the fourth aspect of the present invention.

In a seventh aspect, the present invention provides use of a gel according to the second or third aspects of the present invention in the manufacture of a medicament for treating or preventing a disorder in a subject in need thereof.

In a eighth aspect, the present invention provides use of a pharmaceutical composition according to the fourth aspect of the present invention in the manufacture of a medicament for treating or preventing a disorder in a subject in need thereof.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this specification.

In order that the present invention may be more clearly understood, preferred forms will be described with reference to the following drawings and examples.

### BRIEF DESCRIPTION OF THE DRAWINGS

FIG. 1 shows the relative rates of hyaluronidase digestion using 4.5 mg of an HA gel for a standard 0.075% BDDE cross-linked HA gel; a standard 0.075% BDDE cross-linked HA gel which also contained 0.056% glycidol during manufacture; and a standard 0.075% BDDE cross-linked HA gel which also contained 0.1052% glycidol during manufacture.

FIG. 2 shows the relative rates of hyaluronidase digestion using 4 mg of each of a 0.1% BDDE HA gel; a 1.0% BDDE HA gel; a 0.1% BDDE HA gel manufactured with the addition of 0.9% BDDE epoxide equivalents of glycidol; and commercially available Restylane (Q-Med AB, Seminarregaten 21,SE-752 28 Uppsala, Sweden). Each number given is in comparison to the value obtained for the 0.1% BDDE HA gel and expressed as a ratio.

FIG. 3 shows the relative stress modulus (G′) of a 0.1% BDDE HA gel; a 1.0% BDDE HA gel; a 0.1% BDDE HA gel manufactured with the addition of 0.9% BDDE epoxide equivalents of Glycidol; and commercially available Restylane.

### MODE(S) FOR CARRYING OUT THE INVENTION

### Definitions

As used herein, the term “masking agent” means any mono-functional epoxide capable of chemically modifying the structure of a polysaccharide such that it reduces the ability of an enzyme to recognise and degrade a cross-liked polysaccharide gel through cleavage of the polysaccharide.

As used herein, the term “resistance to degradation under physiological conditions” means conditions of around neutral pH and physiological temperature, preferably pH 7.4 and about 37° C.

As used herein, the term “sufficiently cross-linked to resist degradation” means that the gel is relatively stable to hyaluronidase degradation under physiological conditions over prolonged periods or can tolerate extrusion by being expelled from a small gauge needle.

As used herein, the term “small gauge needle” means a 27, 30 or 32 gauge.

As used herein, the term “alkaline medium” includes, but is not limited to a hydroxide salt dissolved in water, preferably sodium hydroxide.

As used herein, the term “acidic medium” includes, but is not limited to an organic or inorganic acid dissolved in water, preferably acetic acid.

### EXAMPLES

In one embodiment, the present invention provides a process for producing a cross-linked polysaccharide gel. First, a polysaccharide mixed with an alkaline medium is contacted with a cross-linking agent to form an essentially epoxy cross-linked polysaccharide in which the epoxide is linked to the polysaccharide substantially by ether bonds. The epoxy cross-linked polysaccharide is then dried without removing the epoxide from the alkaline medium. The resulting dried cross-linked polysaccharide matrix is then treated with an acidic medium to neutralize the formed cross-linked polysaccharide gel and may then be washed in a suitable water miscible solvent.

### Example 1

### 0.075% BDDE Cross-Linked HA Hydrogel Preparation

Sample of powder hyaluronic acid [Fluka from Streptococcus equi (MW 1.69 MD)] (4.00 g) was dissolved in 1% NaOH (100 ml) with vigorous stirring over a period of 60 minutes at 40° C., 1,4-Butanediol diglycidyl ether (BDDE; 75.0 μl, 0.376 mmol) in THF (425.0 μl) was then added with vigorous stirring and stirring continued for 45 minutes at 40° C. The solution was then dried under high vacuum (30 mbar) for 1.0 hour at 40° C. with slow rotation until weight=7.32 g.

The resulting transparent polysaccharide matrix was rehydrated with acetic acid in water (2.6% v/v; 100 ml) for 20 minutes and the gel was slowly lifted from the glass edges during this time. The pH of the fully swollen gel at the end of this process had been neutralized. Isopropyl alcohol (200 ml) was then added to the gel and the gel Was left to stand for a further 45 minutes with swirling. The IPA/H₂O mixture was decanted off and the gel partially rehydrated with H₂O (100 ml) before IPA (150 ml) was added (IPA/H₂O mixture 6:4) and left to stand for a 45 minutes with swirling. The pH of the filtrate at the end of this process remained neutral. The IPA/H₂O mixture was decanted off and the gel partially rehydrated again with H₂O (50 ml) before IPA (200 ml) was added (IPA/H₂O mixture 8:2) and left to stand for a 30 minutes with swirling. The IPA/H₂O mixture was decanted off and the gel washed with IPA (200 ml) and again left to stand for 15 minutes with swirling. After decanting off the IPA the resulting opaque stiff material was freeze dried over 2 days to give 4.01 g of an opaque white flaky material.

### 0.075% BDDE Cross-Linked HA Hydrogel Preparation With 0.0526% Glycidol

A sample of powdered hyaluronic acid [Fluka from Streptococcus equi (MW 1.69 MD)] (4.00 g) was dissolved in 1% NaOH (100 ml) with vigorous stirring (400 rpm) over a period of 60 minutes at 40° C. 1,4-Butanediol diglycidyl ether (BDDE; 75.0 μl, 0.376 mmol) and Glycidol (52.6 μl, 0.760 mmol) together in THF (372.4 μl) was then added with vigorous stirring (300 rpm) and stirring continued for 45 minutes at 40° C. The solution was then dried under high vacuum (30 mbar) for 1.0 hours at 40° C. with slow rotation until weight=7.14 g.

The resulting transparent polysaccharide matrix was rehydrated with acetic acid in water (2.6% v/v; 100 ml) for 20 minutes and the gel was slowly lifted from the glass edges during this time. The pH of the fully swollen gel at the end of this process had been neutralized. Isopropyl alcohol (200 ml) was then added to the gel and the gel was left to stand for a further 45 minutes with swirling. The IPA/H₂O mixture was decanted off and the gel partially rehydrated with H₂O (100 ml) before IPA (150 ml) was added (IPA/H₂O mixture 6:4) and left to stand for a 45 minutes with swirling. The pH of the filtrate at the end of this process remained neutral. The IPA/H₂O mixture was decanted off and the gel partially rehydrated again with H₂O (50 ml) before IPA (200 ml) was added (IPA/H₂O mixture 8:2) and left to stand for a 30 minutes with swirling. The IPA/H₂O mixture was decanted off and the gel washed with IPA (200 ml) and again left to stand for 15 minutes with swirling. After decanting off the IPA the resulting opaque stiff material was freeze dried over 2 days to give 4.20 g of an opaque white flaky material.

### Swelling Test

Samples (1.00 g) of each of the dry gels were weighed out into screw-top glass jars. Phosphate Buffered Saline (PBS) (80 ml) was then added to each and the gels were left to swell over a period of 72 hours at 20° C. The gels were then blotted to surface dryness on Whatman filters and weighed. There was no visible difference between the gels.

0.075% BDDE Mid-Scale=64.53 g=15.7 mg/ml
0.075% BDDE Mid-Scale with 0.0526% Glycidol=67.84 g=15.0 mg/ml

### Milling and Needle Test

Samples of the above swollen gels were milled through a 212 μm sieve and stored at 0° C. Samples of both milled gels passed easily and similarly through a 32 gauge needle.

### Hyaluronidase Resistance

To determine the concentration of Uronic acid (UA) released by hyaluronidase [EH 3.2.1.35] from the prepared samples the procedure reported by Zhao et al. (Zhao X. B., Fraser J. E., Alexander C., Lockett C. and White B. J. Materials Science, Materials in Medicine 2002, 13, 11-16) was followed essentially identically. In this case assays were developed to measure initial rates of HA release from the gel particle.

Samples (3000 μg) were made up to a final volume of 1 ml in a hyaluronidase solution (containing 0.05 mg/ml hyaluronidase: 1010 units/mg) in PBS pH 7.4. A sample (150 μl) was taken at time 0 hrs and the samples incubated at 37° C. After allotted times samples (150 μl) were removed, centrifuged for 5 minutes and 100 μl placed in 200 μl PBS (pH 7.4). The samples were heated at 100° C. in a heater block for 60 minutes, cooled and stored. Samples for the standard carbazole assay (Bitter T. and Muir H. M. Anal. Biochem. 1962, 4, 330-334) were diluted 10-fold in PBS (pH 7.4) prior to assay. Initial rates were estimated from the rate of release of <400 μg (˜25%) of available uronic acid (˜1500 μg).

### Example 2

### 0.075% BDDE Cross-Linked HA Hydrogel With 0.1052% Glycidol

A sample of powdered hyaluronic acid [Fluka from Streptococcus equi (MW 1.69 MD)] (4.00 g) was dissolved in 1% NaOH (100 ml) with vigorous stirring over a period of 60 minutes at 40° C. 1,4-Butanediol diglycidyl ether (BDDE; 75.0 μl, 0.376 mmol) and Glycidol (105.2 μl, 1.520 mmol) together in THF (319.8 μl) was then added with vigorous stirring and stirring continued for 45 minutes at 40° C. The solution was then dried under high vacuum (30 mbar) for 1.0 hours at 40° C. with slow rotation until weight=7.43 g.

The resulting transparent polysaccharide matrix was rehydrated with acetic acid in water (2.6% v/v; 100 ml) for 20 minutes and the gel was slowly lifted from the glass edges during this time. The pH of the fully swollen gel at the end of this process had been neutralized. Isopropyl alcohol (200 ml) was then added to the gel and the gel was left to stand for a further 45 minutes with swirling. The IPA/H₂O mixture was decanted off and the gel partially rehydrated with H₂O (100 ml) before IPA (150 ml) was added (IPA/H₂O mixture 6:4) and left to stand for a 45 minutes with swirling. The pH of the filtrate at the end of this process remained neutral. The IPA/H₂O mixture was decanted off and the gel partially rehydrated again with H₂O (50 ml) before IPA (200 ml) was added (IPA/H₂O mixture 8:2) and left to stand for a 30 minutes with swirling. The IPA/H₂O mixture was decanted off and the gel washed with IPA (200 ml) and again left to stand for 15 minutes with swirling. After decanting off the IPA the resulting opaque stiff material was freeze dried over 2 days to give 4.19 g of an opaque white flaky material.

### Swelling Test

Samples (1.00 g) of each of the dry gels were weighed out into screw-top glass jars. PBS (80 ml) was then added to each and the gels were left to swell over a period of 72 hours at 20° C. The gels were then blotted to surface dryness on Whatman filters and weighed.

0.075% BDDE Mid-Scale with 0.1052% Glycidol=49.57 g=20.6 mg/ml
20.6 mg/ml

### Milling and Needle Test

Samples of the above swollen gels were milled through a 212 μm sieve and stored at 0° C. Samples of both milled gels passed easily through a 32 gauge needle.

### Hyaluronidase Resistance

To determine the concentration of Uronic acid (UA) released by hyaluronidase [EH 3.2.1.35] from the prepared sample the procedure reported by Zhao et al. (Zhao X. B., Fraser J. E., Alexander C., Lockett C. and White B. J. Materials Science, Materials in Medicine 2002, 13, 11-16) was followed essentially identically. In this case assays were developed to measure initial rates of HA release from the gel particle.

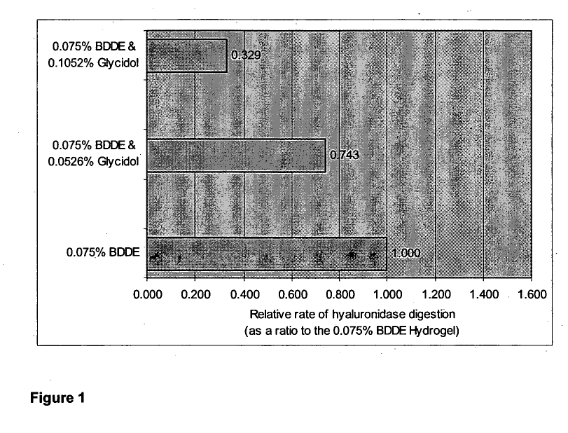

Samples (4500 μg) were made up to a final volume of 1.5 ml in a hyaluronidase solution (containing 0.01 mg/ml hyaluronidase: 1010 units/mg) in phosphate buffered saline (PBS, pH 7.4). A sample (150 μl) was taken at time 0 hrs and the samples incubated at 37° C. After allotted times samples (150 μl) were removed and added to 300 μl PBS at 0° C. and centrifuged for 5 minutes. Then 200 μl was placed in a new sample tube being careful to avoid any pelleted material. The samples were then heated at 100° C. in a heater block for 60 minutes, cooled and stored. Samples for the standard carbazole assay (Bitter T. and Muir H. M. Anal. Biochem. 1962, 4, 330-334) were diluted 5-fold in PBS prior to assay (FIG. 1).

### Example 3

### 0.1% BDDE HA Hydrogel Preparation

A sample of soluble powdered sodium hyaluronate [Fluka from Streptococcus equi (MW 1.69 MD)] (2.0000 g) was dissolved in a solution of 1% w/v NaOH (50 ml) by mixing with vigorous stirring over a period of 20 minutes at 40° C. Fresh 1,4-butanediol diglycidyl ether (BDDE; 47.9 mg, 0.225 mmol) was then added dropwise and the solution was stirred for 20 minutes at 40° C. The solution was then dried under vacuum for 30 minutes at 40° C. whilst rotating the reaction flask. During this time the evaporation was carefully manipulated such that the body of viscous liquid was deposited evenly over the inside surface of the barrel of reaction flask used. This was continued until the total weight of the H₂O in the reaction was approximately equal to that of the original weight of HA.

The resulting polysaccharide matrix was left to stand for 20 minutes in the dry state at room temperature. The gel was then partially rehydrated and neutralized. with acetic acid in water (2.6% v/v, 50 ml) for 5 minutes whilst standing still and the gel was then lifted from the glass as single sheet. Rehydration was then continued for a further 15 minutes. Isopropyl alcohol (IPA; 200 ml) was then added to the gel (final IPA/H₂O mixture 4:1) and the gel was swirled gently over 30 minutes. The IPA/H₂O mixture was decanted off. The gel was then partially rehydrated with H₂O (100 ml) for 15 minutes at room temperature whilst standing still. IPA (400 ml) was then added (final IPA/H₂O mixture 4:1) and left to stand for 30 minutes with swirling as before. The IPA/H₂O mixture was decanted off. Some of the remaining IPA was removed by evaporation at the vacuum pump for 15 minutes at 35° C.

The gel was then partially rehydrated with H₂O to a concentration of HA of approximately 15 mg/ml. The gel was left to stand for 20 minutes at room temperature. The gel was then chopped into pieces and transferred into cellulose membrane dialysis tubing and dialyzed against stirred deionised water (2000 ml) for 3 hours. The dialysis tubes were removed to fresh deionised water (2000 ml) and stirred over 64 hours at room temperature. The dialysis tubes were removed to fresh deionised water (2000 ml) and stirred over 3 hours at room temperature.

The gel was then dried over a dry nitrogen stream for 36 hours to give a wispy spun sugar-like appearance. The gel was then swollen to 55 mg/ml (based on the recovered dry weight) in sterile PBS for 1 hour at room temperature. A sample of the gel was then milled thrice through a 125 micron sieve and then diluted to 20 mg/ml with sterile PBS. The sample was then sealed and sterilized in an autoclave (121° C. at 1.2 bar for 15 minutes, then 100° C. at 0 bar for 10 minutes). At the end of the cycle the sample was quickly removed from the autoclave and cooled in water at room temperature.

### Example 4

### 1.0% BDDE HA Hydrogel Preparation

A sample of soluble powdered sodium hyaluronate [Fluka from Streptococcus equi (MW 1.69 MD)] (2.0000 g) was dissolved in a solution of 1% w/v NaOH (50 ml) by mixing with vigorous stirring over a period of 20 minutes at 40° C. Fresh 1,4-butanediol diglycidyl ether (BDDE; 478.5 mg, 2.248 mmol) was then added dropwise and the solution was stirred for 20 minutes at 40° C. The solution was then dried under vacuum for 30 minutes at 40° C. whilst rotating the reaction flask. During this time the evaporation was carefully manipulated such that the body of viscous liquid was deposited evenly over the inside surface of the barrel of reaction flask used. This was continued until the total weight of H₂O in the reaction was approximately equal to that of the original weight of HA.

The resulting polysaccharide matrix was left to stand for 20 minutes in the dry state at room temperature. The gel was then partially rehydrated and neutralized with acetic acid in water (2.6% v/v, 50 ml) for 5 minutes whilst standing still and the gel was then lifted from the glass as single sheet. Rehydration was then continued for a further 15 minutes. Isopropyl alcohol (IPA; 200 ml) was then added to the gel (final IPA/H₂O mixture 4:1) and the gel was swirled gently over 30 minutes. The IPA/H₂O mixture was decanted off. The gel was then partially rehydrated with H₂O (100 ml) for 15 minutes at room temperature whilst standing still. IPA (400 ml) was then added (final IPA/H₂O mixture 4:1) and left to stand for 30 minutes with swirling as before. The IPA/H₂O mixture was decanted off. Some of the remaining IPA was removed by evaporation at the vacuum pump for 15 minutes at 35° C.

The gel was then partially rehydrated with H₂O to a concentration of HA of approximately 30 mg/ml. The gel was left to stand for 20 minutes at room temperature. The gel was then chopped into pieces then fully rehydrated with deionised H₂O (to a volume of 2000 ml) for 3 hours at room temperature during which time the gel was gently swirled. The water was decanted off under a slight vacuum over a 11 micron nylon mesh covered sinter to collect the gel. Then 500 ml fresh deionised water was added. This was left for a 20 minutes at room temperature and the water again decanted off under a slight vacuum over a 11 micron nylon mesh covered sinter to collect the gel. Then the gel was made up to a volume of 2000 ml with fresh deionised water and left over night (16 h) at room temperature during which time the gel was gently swirled. The water was again decanted off under a slight vacuum over a 11 micron nylon mesh covered sinter to collect the gel. Then 1000 ml fresh deionised water was added. This was left for a 3 hours at room temperature and the water again decanted off under a slight vacuum over a 11 micron nylon mesh covered sinter to collect the gel.

The gel was then dried over a dry nitrogen stream for 48 hours to give a wispy spun sugar-like appearance. The gel was then swollen to 55 mg/ml (based on the recovered dry weight) in sterile PBS for 1 hour at room temperature. A sample of the gel was then milled thrice through a 125 micron sieve and then diluted to 20 mg/ml with sterile PBS. The sample was then sealed and sterilized in an autoclave (121° C. at 1.2 bar for 15 minutes, then 100° C. at 0 bar for 10 minutes). At the end of the cycle the sample was quickly removed from the autoclave and cooled in water at room temperature.

### Example 5

### 0.1% BDDE and 0.9% Glycidol HA Hydrogel Preparation

A sample of soluble powdered sodium hyaluronate [Fluka from Streptococcus equi (MW 1.69 MD)] (2.0000 g) was dissolved in a solution of 1% w/v NaOH (50 ml) by mixing with vigorous stirring over a period of 20 minutes at 40° C. At this point the solution was clear. Fresh 1,4-butanediol diglycidyl ether (BDDE; 47.9 mg, 0.225 mmol) was then added dropwise and the solution was stirred for 18 minutes at 40° C. Fresh glycidol (299.7 mg, 4.046 mmol) was then added dropwise and the solution was stirred for 2 minutes at 40° C. The solution was then dried under vacuum for 30 minutes at 40° C. whilst rotating the reaction flask. During this time the evaporation was carefully manipulated such that the body of viscous liquid was deposited evenly over the inside surface of the barrel of reaction flask used. This was continued until the total weight of H₂O in the reaction was approximately equal to that of the original weight of HA.

The resulting polysaccharide matrix was left to stand for 20 minutes in the dry state at room temperature. The gel was then partially rehydrated and neutralized with acetic acid in water (2.6% v/v, 50 ml) for 5 minutes whilst standing still and the gel was then lifted from the glass as single sheet. Rehydration was then continued for a further 15 minutes. Isopropyl alcohol (IPA; 200 ml) was then added to the gel (final IPA/H₂O mixture 4:1) and the gel was swirled gently over 30 minutes. The IPA/H₂O mixture was decanted off. The gel was then partially rehydrated with H₂O (100 ml) for 15 minutes at room temperature whilst standing still. IPA (400 ml) was then added (final IPA/H₂O mixture 4:1) and left to stand for 30 minutes with swirling as before. The IPA/H₂O mixture was decanted off. Some of the remaining IPA was removed by evaporation at the vacuum pump for 15 minutes at 35° C.

The gel was then partially rehydrated with H₂O to a concentration of HA of approximately 15 mg/ml. The gel was left to stand for 20 minutes at room temperature. The gel was then chopped into pieces and transferred into cellulose membrane dialysis tubing and dialyzed against stirred deionised water (2000 ml) for 1.5 hours. The dialysis tubes were removed to fresh deionised water (2000 ml) and again stirred over 1.5 hours at room temperature. The dialysis tubes were removed to fresh deionised water (2000 ml) and stirred over 16 hours at room temperature.

The gel was then dried over a dry nitrogen stream for 32 hours to a wispy spun sugar-like appearance. The gel was then swollen to 55 mg/ml (based on the recovered dry weight) in sterile PBS for 1 hour at room temperature. A sample of the gel was then milled thrice through a 125 micron sieve and then diluted to 20 mg/ml with sterile PBS. The sample was then sealed and sterilized in an autoclave (121° C. at 1.2 bar for 15 minutes, then 100° C. at 0 bar for 10 minutes). At the end of the cycle the sample was quickly removed from the autoclave and cooled in water at room temperature.

### Hyaluronidase Resistance

To determine the concentration of released N-acetyl glucosamine by hyaluronidase [EH 3.2.1.35] from the prepared samples the procedure reported by Reissig et al. (Reissig J. L, Strominger J. L, and Leloir L. F, A modified colorimetric method for the estimation of N-acetylaminosugars, J. Biol. Chem. 1955, 217 (2), 959-966) was followed with adjustments.

Identical twin samples of exactly 4 mg of HA (dry weight calculated from that obtained after extensive drying of the dialysed gel during manufacture or as given on the box for Restylane) extruded through a 30 G needle were placed into eppendorf tubes and made up to 0.700 ml with phosphate buffered saline (PBS, pH 7.20) and the mix vortexed to an even suspension. The suspensions were then incubated at 37° C. for 10 minutes prior to the addition of enzyme. To each of the identical twin solutions was added either PBS (100 μl) or enzyme (100 μl) containing 0.1 mg/ml hyaluronidase (bovine testes type IV-S; 1010 units/mg solid) in PBS and each was vortexed. The samples were then incubated at 37° C. for 16 hrs. From each of the assay reaction mixes, 200 μl was added to 50 μl potassium tetraborate solution (0.4 mol/l; pH 9.1). These were then used directly in the colour assay.

To these samples (200 μl) was added 1.2 ml of diluted Ehrlich's solution. Samples were then heated at 37° C. for 30 minutes. The samples were then centrifuged for 5 minutes to pellet non digested material and the absorbance measured at 585 nm. In each case a blank sample containing 200 μl of PBS and 50 μl potassium tetraborate solution (0.4 mol/l; pH 9.1) was prepared to zero the spectrometer. The average reading obtained for three identical assay samples without added enzyme was then subtracted from the average reading obtained for three identical assay samples with added enzyme.

### Rheology

Samples of gels extruded through a 30 G needle were measured using a Parr rheometer (MCR301 SN80108726) with parallel plates (amplitude gamma=1E-3 1E+3% log, slope=6 Pt./dec, frequency 5 Hz, 25° C., distance 0.3 mm). In each case the storage modulus (G′, Pa) was recorded after the normal force had stabilized.

Preferably, the polysaccharide is selected from hyaluronic acid, chondroitin sulphate, heparin, starch, maltodextrins, cellodextrins, cellulose, chitosan, glucomannan, pectin, xanthan, algiinic acid, carboxymethyl cellulose, carboxymethyl dextran, carboxymethyl starch and carrageenans. More preferably the polysaccharide is hyaluronic acid.

Preferably the reaction is carried out with concentrations of the polysaccharide within the range of about 0.1 to 10% (w/v). More preferably the reaction is carried out with the concentration of the polysaccharide within the range of about 3 to 6% (w/v). Most preferably, the reaction is carried out with the concentration of the polysaccharide being about 4% (w/v).

Preferably the reacted gels may be formulated into gels for injection containing the polysaccharide within the range of about 0.1 to 100 mg/ml. More preferably, the reacted gels may be formulated into gels for injection containing the polysaccharide within the range of about 5 to 50 mg/ml. Most preferably, the reacted gels may be formulated into gels for injection containing the polysaccharide within the range of about 10 to 40 mg/ml.

### Uses of Gels

Hyaluronic acid gels may be injected into the epidermis, dermis, subcutaneous tissues or supra-periostial tissues to augment and provide greater volume to these tissues in cases of tissue loss due to ageing or trauma, infection, acne or any other disease. The gels may be injected into vocal folds to enhance their function when function is impaired. The gels may be injected into peri-urethral tissues as a treatment for urethral incontinence. The gels may be injected into any bodily soft tissue which might require augmentation of volume. The gels may be injected into cartilaginous joints in cases of arthritis to improve function and decrease pain. The gels may be injected into the intra-abdominal cavity to impair or prevent the formation of adhesions due to surgery or disease. The gels may be injected into the eyes to replace vitreous humor, for example, during surgery to the eyes. Moreover, the gels may also be used in the treatment of arthritis. Depending upon the use and the viscosity of the gels, they may be injected through cannulas or needles in size from 10 gauge to 33 gauge in size.

Gels arising from the present invention may contain concentrations of cross-linked polysaccharides modified to resist in vivo degradation previously not able to be administered by injection or cannula because of their viscosity. Additionally, concentrations of polysaccharides modified to resist in vivo degradation currently able to be administered by injection or cannula may be manufactured using this invention with rheological qualities which will enable administration through finer gauge needles or cannulas, resulting in less trauma and pain. The gels produced by the present invention will maintain longer biological effects than gels manufactured using prior art, resulting in the necessity for fewer treatments and greater utility than gels made using prior art.

### SUMMARY

The assay technique in which the presence of uronic acid is detected provides a satisfactory method of determining the rate of release of soluble hydrogel fragments from formed particulate cross-linked hydrogels. In the case of Examples 1 and 2 where a 0.075% BDDE cross-linked gel (0.376 mmol BDDE; equivalent to 0.752 mmol epoxide) was made with or without the addition of glycidol (at 0.760 mmol and 1.520 mmol equivalents of epoxide), it is apparent that the addition of glycidol markedly improves the resistance of the formed hydrogel to hyaluronidase degradation of this type (see FIG. 1). Furthermore, simple analyses of the swelling capacity of these manufactured gels demonstrated that they most likely contained not dissimilar levels of cross-linking.

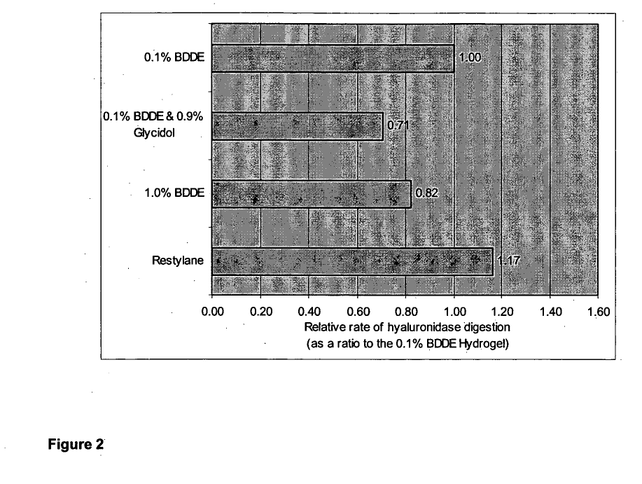

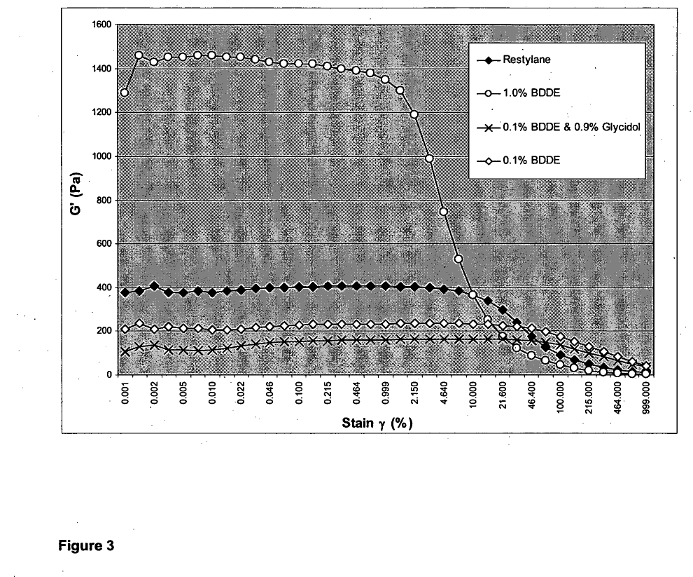

A more effective assay technique for directly determining the activity of hyaluronidase on each formed gel is obtained from that in which the presence of terminal N-acetyl D-glucosamine units are detected. In the case of Examples 3, 4 and 5 where a 0.1% BDDE cross-linked gel (0.225 mmol BDDE; equivalent to 0.45 mmol epoxide), a 1.0% BDDE cross-linked gel (2.248 mmol BDDE; equivalent to 4.496 mmol epoxide), and a 0.1% BDDE cross-linked gel (0.225 mmol BDDE; equivalent to 0.45 mmol epoxide) manufactured in the presence of 0.9% glycidol (4.046 mmol glycidol; equivalent to 4.046 mmol epoxide giving a combined total with the BDDE of 4.496 equivalents of epoxide) it is apparent that the addition of glycidol also markedly improves the resistance of the formed hydrogel to hyalurohidase degradation of this type (see FIG. 2) even after sterilization. Moreover, the addition of the reactive epoxide masking agent did not impact the Theological properties of the formed gel. In this case the relative stress modulus (G′) for the 0.1% BDDE cross-linked hydrogel manufactured with the addition of glycidol demonstrated Theological properties similar to that observed for the 0.1% BDDE cross-linked hydrogel and hyaluronidase resistance similar to that of the 1.0% BDDE cross-linked hydrogel (FIG. 3).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1.-28. (canceled)

29. A process for preparing a cross-linked polysaccharide gel having resistance to enzymatic degradation under physiological conditions, the process comprising:
contacting a polysaccharide with a cross-linking agent under conditions to form a cross-linked polysaccharide gel, and
contacting the polysaccharide with a masking agent selected from the group consisting of a monofunctional epoxide and an alkyl vinyl sulfone;
so as to form a cross-linked polysaccharide gel having resistance to enzymatic degradation.

30. The process according to claim 29, wherein the contacting with a cross-linking agent and the contacting with a masking agent occur substantially at the same time.

31. The process according to claim 29, wherein the contacting with a masking agent occurs after the contacting with a cross-linking agent.

32. The process according to claim 29, wherein the polysaccharide is contacted with the cross-linking agent under alkaline conditions to form a cross-linked polysaccharide substantially cross-linked by ether bonds.

33. The process according to claim 29, further comprising:
drying the cross-linked polysaccharide without substantially removing the cross-linking agent or the masking agent to form a cross-linked polysaccharide matrix; and
neutralising the cross-linked polysaccharide matrix with an acidic medium to form the cross-linked polysaccharide gel.

34. The process according to claim 29, wherein the polysaccharide is selected from the group consisting of hyaluronic acid, chondroitin sulphate, heparin, maltodextrins, cellodextrins, cellulose, chitosan, glucomannan, pectin, xanthan, algiinic acid, carboxymethyl cellulose, carboxymethyl dextran, and carrageenans.

35. The process according to claim 34, wherein the polysaccharide is hyaluronic acid.

36. The process according to claim 29, wherein the cross-linking agent is selected from the group consisting of aldehydes, epoxides, glycidyl ethers, polyaziridyl compounds, and divinylsulfones.

37. The process according to claim 36, wherein the cross-linking agent is selected from the group consisting of ethylene glycol diglycidyl ether, 1,4-butanediol diglycidyl ether, 1.4-bis(2,3-epoxypropoxy)butane, 1,4-bisglycidyloxybutane, 1,2-bis(2,3-epoxypropoxy)ethylene or 1-(2,3-epoxypropyl)-2,3-epoxycyclohexane.

38. The process according to claim 37, wherein the cross-linking agent is 1,4-butanediol diglycidyl ether.

39. The process according to claim 29, wherein the masking agent is selected from the group consisting of ethylene oxide, propylene oxide, ethyl vinyl sulfone, methyl vinyl sulfone, and glycidol.

40. The process according to claim 39, wherein the masking agent is glycidol or ethyl vinyl sulfone.

41. The process according to claim 40, wherein the masking agent is glycidol.

42. The process according to claim 39, wherein the cross-linking agent and masking agent are used under alkaline conditions.

43. The process according to claim 32, wherein the alkaline conditions have a pH in the range of about 8 to 14.

44. The process according to claim 42, wherein the alkaline conditions have a pH in the range of about 8 to 14.

45. The process according to claim 32, wherein the alkaline conditions are formed by from 0.1 to 1 w/v percent of NaOH or KOH.

46. The process according to claim 29, wherein the step of contacting the polysaccharide with the cross-linking agent comprises between 1 and 10 w/v percent polysaccharide and between 0.05 and 1.0 w/v percent cross-linking agent.

47. The process according to claim 46, comprising about 4 w/v percent polysaccharide.

48. The process according to claim 47, comprising about 0.1 w/v percent cross-linking agent.

49. The process according to claim 29, wherein each contacting step is carried out at a temperature in the range 0-100° C.

50. The process according to claim 49, wherein each contacting step is carried out at a temperature of at least about 40° C.

51. The process according to claim 29, wherein the cross-linked polysaccharide is dried.

52. The process according to claim 33, wherein the acidic medium comprises acetic acid or hydrochloric acid.

53. The process according to claim 29, further comprising:
washing the cross-linked polysaccharide gel with a water-miscible solvent.

54. The process according to claim 53, wherein the water-miscible solvent is isopropyl alcohol.

55. The process according to claim 33, wherein following the neutralisation step the cross-linked polysaccharide gel is dried and optionally reconstituted.

56. The process according to claim 55, wherein the dried cross-linked polysaccharide gel is reconstituted in phosphate buffered saline.

57. A method of augmenting tissue comprising administering to a subject a cross-linked polysaccharide gel prepared by the process of claim 29.

58. A method of treating a subject in need thereof, comprising administering to the subject an effective amount of a cross-linked polysaccharide gel prepared by the process of claim 29.
